(19) United States
(12) Patent Application Publication
(10) Pub. No.: US 2011/0313239 A1
(43) Pub. Date: Dec. 22, 2011

(54) METHODS AND DEVICES FOR GENERATING BREATH CUES

(76) Inventor: Adam Jude Ahne, Carbondale, IL (US)

(21) Appl. No.: 12/817,131

(22) Filed: Jun. 16, 2010

(51) Int. Cl. A61M 21/00

(52) U.S. Cl. 600/27

(57) ABSTRACT

A method of generating light is disclosed. The method includes generating steady light during a first period. The method also includes generating varying light during a second period, the second period is less than one quarter of the first period, and the varying light is in the form of breath cues. Other methods and devices are disclosed.

### FIELD

The present disclosure relates to methods and devices for generating breath cues.

### BACKGROUND

This section provides background information related to the present disclosure which is not necessarily prior art.

The benefits of controlled breathing exercises are known. Controlled breathing reduces stress and allows for greater concentration. Known devices and software programs provide breathing cues that an observer may use to time controlled breathing. However, known devices may be difficult to incorporate into daily routines because the devices either provide a continuous stream of breathing cues, which may be eventually tuned-out by a user, or the devices must be triggered by a user to initiate each session of controlled breathing, which may lead to the device being ignored.

### SUMMARY

This section provides a general summary of the disclosure, and is not a comprehensive disclosure of its full scope or all of its features.

According to one example embodiment of the present disclosure, a method of generating light is disclosed. The method includes generating steady light during a first period. The first period is at least twenty minutes and is less than twelve hours. The method also includes generating varying light during a second period, the second period is less than one quarter of the first period, and the varying light is in the form of breath cues.

According to another example embodiment of the present disclosure, a device configured to: (a) generate breath cues during a first period, the first period is between one minute and thirty minutes, the breath cues are such that the total time for a complete inhale/exhale breath cycle is between six and twenty seconds; (b) not generate breath cues during a second period, the second period is between thirty minutes and four hours; and repeat (a) and (b).

According to yet another example embodiment of the present disclosure, a device includes means for generating steady light during a first period, the first period is at least twenty minutes; and means for generating varying light during a second period, the second period is less than one quarter of the first period, the varying light is in the form of breath cues.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 shows a graph of light intensity versus time according to an example embodiment of the present disclosure.

FIG. 2 shows a graph of light intensity versus time according to another example embodiment of the present disclosure.

FIG. 3 shows a graph of light intensity versus time according to another example embodiment of the present disclosure.

FIG. 4 shows a diagram of a lamp according to another example embodiment of the present disclosure.

FIG. 5 shows a diagram of a prompter according to another example embodiment of the present disclosure.

FIG. 6 shows a schematic of a device according to another example embodiment of the present disclosure.

FIG. 7 shows a schematic of a device according to another example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used in the present disclosure, a breath cue is an indicator of when an observer should breathe in or breathe out. A breath cue has an observable beginning, an observable ending, or both, so that an observer knows when to begin breathing in or breathing out. Preferably, a breath cue warns an observer of the approaching end of the breath cue so the observer is ready to react to the end of the breath cue, react to the beginning of the next breath cue, etc.

An observer synchronizing their breathing to a series of breath cues will complete an inhale/exhale breath cycle over two breath cues. Preferably, the duration of each breath cue is about five seconds so that the observer completes a breath cycle every ten seconds i.e. 0.1 Hz. The inventor has observed that breathing at about 0.1 Hz causes the observer's pulse rate to synchronize with their breathing and tends to encourage a state of relaxation.

According to one example embodiment of the present disclosure, a method of generating light is disclosed. The method includes generating steady light during a first period. The first period is at least twenty minutes and is less than twelve hours. The method also includes generating varying light during a second period, the second period is less than one quarter of the first period, and the varying light is in the form of breath cues.

The method may be performed at the beginning of a study session. In an example embodiment, the method may begin with a period of breath cues to relax the observer and help them prepare to study. Next is a period of steady light which is helpful for studying. Alternatively, the period of steady light may occur first and the breath cues may prompt the observer to take a break by performing controlled breathing.

As used in the present disclosure, steady light means light that is perceived as steady by the human eye. For example, fluorescent lamps turning on and off at an AC mains frequency (50 or 60 Hz) generate steady light.

The method may repeat the steady light period and the varying light period. This will prompt an observer to occasionally perform controlled breathing.

Preferably, each breath cue has the same duration and the duration is about five seconds. In this example, since each breath cue is the same the observer does not have to worry about distinguishing an inhale breath cue from an exhale breath cue. A five second breath cue will give a 0.1 Hz breath cycle. Of course, the duration of sequential breath cues could be different and the duration of breath cues could vary during the second period.

The method may further include generating an end-of-cycle indication after the last breath cue of the second period. This instructs the observer to, for example, end the controlled breathing and resume working. The end-of-cycle indication may take the form of, for example, a light intensity, no light, a noise, etc.

Further, it is preferable that the second period does not contain a period of generating no light that is longer than one thirtieth of a second. Some observers find darkness unsettling and anxiety provoking.

Generating varying light may include generating light of varying colors. For example, a first color may be generated during a first breath cue and a second color may be generated during a second breath cue. The transition from a first color to a second color may signify a transition between sequential breath cues.

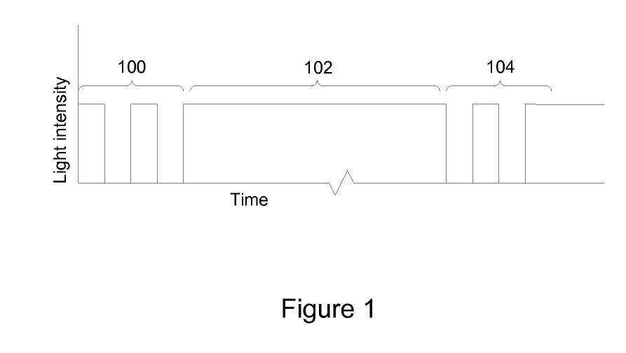

FIG. 1 shows an example embodiment of the present disclosure. A series of breath cues 100 is followed by a period of steady light 102 which is followed by a series of breath cues 104.

In this example embodiment, each breath cue is either 100% light intensity or 0% light intensity (i.e. generating no light). Alternatively, a breath cue may include a period of 100% light intensity and a period of 0% light intensity.

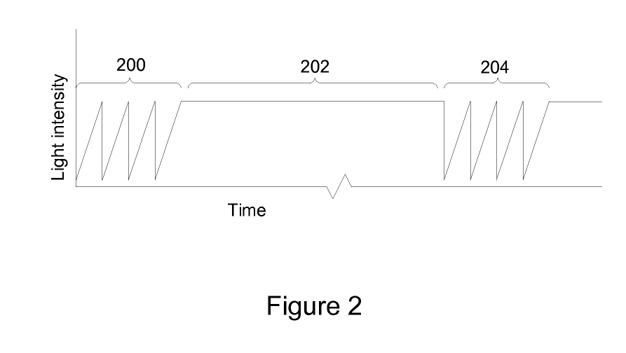

FIG. 2 shows another example embodiment of the present disclosure. A series of breath cues 200 is followed by a period of steady light 202 which is followed by a series of breath cues 204.

In this example embodiment, sequential breath cues are the same. Each breath cue includes a plurality of light intensities. For example, the breath cue light intensity begins at 10% and ramps linearly to 100% at the end of each breath cue. In this example, the light intensity ramps continuously. Alternatively, the light intensity may ramp in discrete steps. Of course, other intensity profiles may be used, e.g. parabolic, sinusoidal, etc. and breath cues may have different profiles.

It is preferable to begin the breath cue with a lower light intensity than the ending so that the transition between breath cues is a sudden dimming instead of a sudden brightening. A sudden dimming is less disturbing to an observer and helps the observer maintain a state of relaxation. Alternatively, the breath cue may begin with a higher light intensity than the ending.

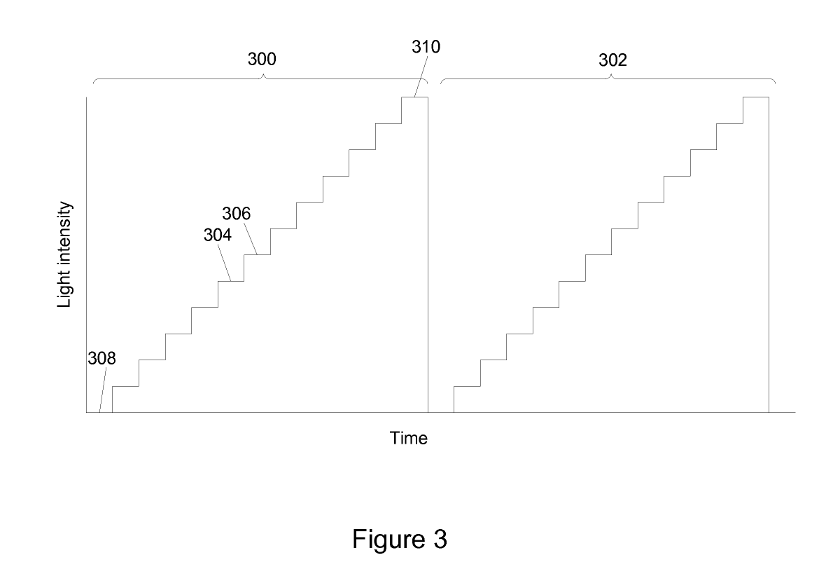

FIG. 3 shows another example embodiment of the present disclosure. A first breath cue 300 and a second breath cue 302 include periods of discrete light intensity (e.g. period 304 and period 306). In this example embodiment, the first breath cue 300 and the second breath cue 302 are the same. The first breath cue 300 begins with a period of no light 308 and ends with a period of greatest light intensity 310.

Each period of discrete light intensity may be long enough for an observer to count. For example, each period of discrete light intensity may be between a third of a second and three seconds. By counting these sub-periods, the observer can determine how much time remains in each breath cue. This may make it easier for the observer to synchronize their breathing to the breath cues.

Note that the light intensity may represent continuous light. Alternatively, the light intensity may represent pulse-width-modulated light at a frequency above the response rate of the human eye, e.g. 200 Hz, etc.

According to another example embodiment of the present disclosure, a device configured to: (a) generate breath cues during a first period, the first period is between one minute and thirty minutes, the breath cues are such that the total time for a complete inhale/exhale breath cycle is between six and twenty seconds; (b) not generate breath cues during a second period, the second period is between thirty minutes and four hours; and repeat (a) and (b).

The device may include one or more of the following breath cue generators: a light, a series of lights, multiple colors of lights, a speaker, a vibrator, etc. A complete inhale/exhale breath cycle may comprise two breath cues.

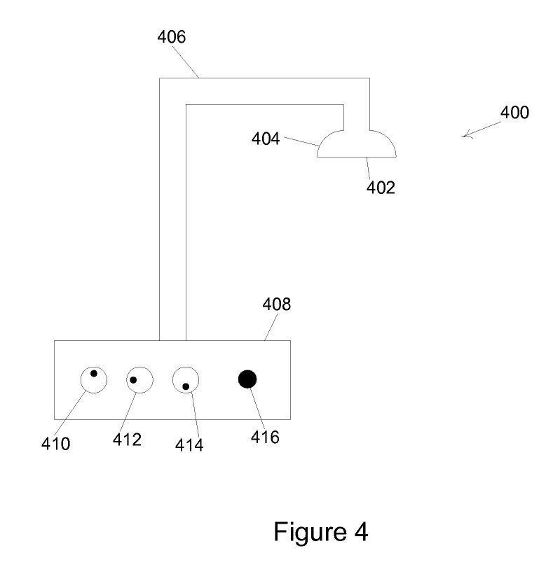

FIG. 4 shows an example embodiment of the present disclosure. A lamp 400 includes a light emitter 402, a head 404, an arm 406, and a base 408. The light emitter 402 is housed within the head 404. The arm 406 connects the head 404 to the base 408.

The base includes a first knob 410, a second knob 412, a third knob 414, and a button 416. Alternatively, any one or more of the first knob 410, the second knob 412, the third knob 414, and the button 416 may be included in the head 404.

The light emitter 402 may include one or more incandescent bulbs, fluorescent bulbs, halogen bulbs, light emitting diodes, etc.

The lamp 400 also includes a controller (not shown) configured to perform one or more methods of the present disclosure. The controller may be located, for example, in the base 408, the head 404, etc. In other embodiments of the present disclosure, the controller is physically separate from the lamp 400.

In this example embodiment, the controller is configured to adjust the duration of a period of steady light based on the position of the first knob 410. Other user controls are envisioned, such as switches, sliders, etc. Similarly, the controller is configured to adjust the duration of a period of varying light that includes breath cues based on the position of the second knob 412. The controller is further configured to adjust the duration of each breath cue based on the position of the third knob 414.

The controller is further configured to exit generating steady light and begin generating breath cues when the button 416 is pressed. This may be used by an observer who wishes to immediately begin controlled breathing. Alternatively, the controller may be configured to exit generating breath cues and begin generating steady light when the button 416 is pressed. This may be used by an observer who wishes to continue working without a controlled breathing session. Alternatively, the controller may be configured to toggle between generating steady light and generating breath cues when the button 416 is pressed.

The controller may be configured to synchronize one or more of its periods to an external signal, such as, for example, a radio wave. This would aid multiple controllers to operate synchronously in installations where one observer may view multiple lamps. Alternatively, the controller may include a real time clock and may be configured to synchronize one or more of its periods to the real time clock.

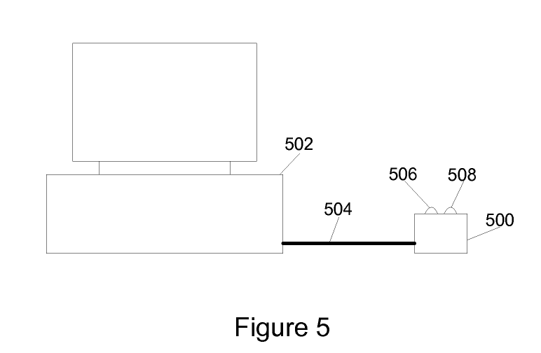

FIG. 5 shows an example embodiment of the present disclosure. A prompter 500 is connected to a computer 502 via a cable 504. The prompter includes a green light emitting diode (LED) 506 and a blue light emitting diode 508. The prompter includes a controller (not shown) coupled to the green light emitting diode 506 and the blue light emitting diode 508.

In this example embodiment, the cable 504 provides power to the prompter 500. The cable may be, for example, a USB cable, etc. The controller may receive configuration information from the computer 502 via the cable 504, e.g. duration of breath cues, etc.

In operation, the prompter 500 generates breath cues by alternating lighting the green LED 506 and the blue LED 508 every five seconds for three minutes. Afterwards, the prompter 500 turns off both the green LED 506 and the blue LED 508 for one hour. The prompter repeats the cycle indefinitely.

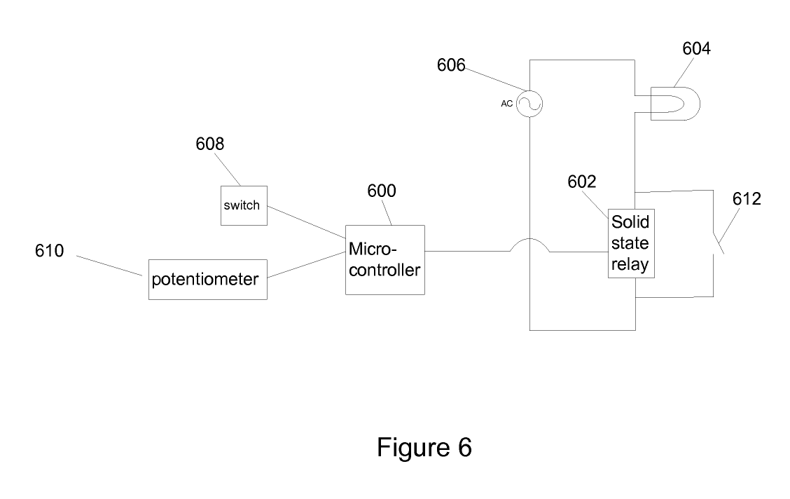

FIG. 6 shows an example embodiment of the present disclosure. A microcontroller 600 is coupled to a solid-state relay 602 to control an incandescent bulb 604 driven by an AC mains 606.

The microcontroller 600 is configured to perform one or more methods of the present disclosure. For example, the microcontroller 600 is configured to generate steady light during a first period and generate varying light during a second period. In this example embodiment, the microcontroller 600 executes a control program. For example, the microcontroller 600 may be a Microchip PIC12F675 microcontroller operating with a 8 kHz oscillator. Alternatively, the microcontroller 600 may be replaced by an application specific integrated circuit configured to perform one or more methods of the present disclosure.

The microcontroller 600 is configured to switch the solid-state relay 602 with a pulse-width-modulated (PWM) drive signal with a base frequency that is synchronized to the AC mains 606 at, for example, 120 Hz. The PWM frequency may alternatively be, for example, 200 Hz, etc. Alternatively, the microcontroller 600 may be configured to drive the solid-state relay 602 with a non-pulse-width-modulated drive signal.

The microcontroller 600 is coupled to a switch 608 and a potentiometer 610. The microcontroller 600 is configured to adjust the duration of a period of generating steady light based on the potentiometer 610. In this example embodiment, the duration is increased as the potentiometer resistance increases. Alternatively, the microcontroller 600 may be configured to adjust a period of generating steady light based on the switch 608. For example, the switch 608 may select between discrete durations.

The incandescent bulb 604 may be replaced with, for example, one or more LEDs with an appropriate power supply in place of the AC mains 606.

An override switch 612 is connected in parallel with the solid state relay 602. The override switch 612 forces the incandescent bulb 604 to remain on regardless of the state of the solid state-relay 602. The override switch 612 may be, for example, a three position switch that also controls system power with the three states of: off, on with steady light, on with periods of steady light and periods of breath cues.

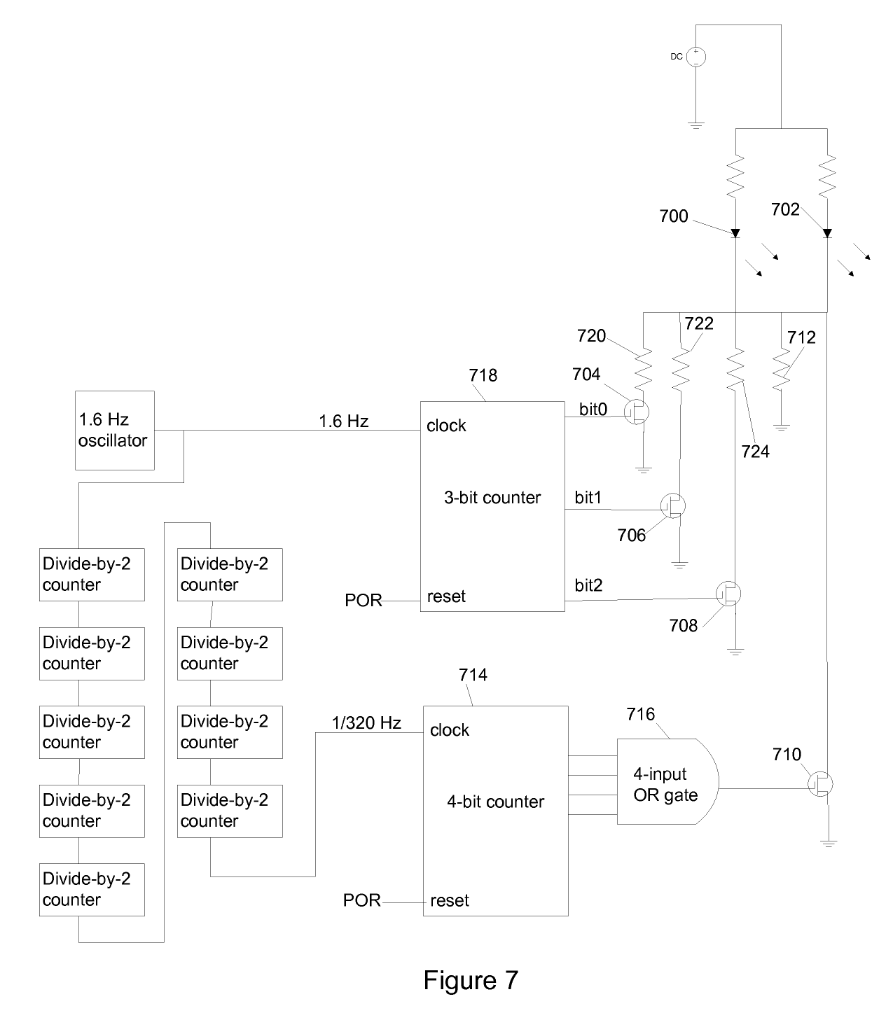

FIG. 7 shows an example embodiment of the present disclosure. A plurality of LEDs 700, 702 generate light based on transistors 704, 706, 708, 710 and a resistor 712. The resistor 712 insures that the LEDs 700, 702 always generate a minimum amount of light.

Transistor 710 is controlled by a 4-bit counter 714 fed by a 1/320 Hz clock. Transistor 710 will be turned off by an OR-gate 716 for 320 seconds and will be turned on for 4,800 seconds. While transistor 710 is on the LEDs 700, 702 will generate steady light since transistor 710 shorts across transistor 704, transistor 706, and transistor 708.

Transistor 704, transistor 706, and transistor 708 are controlled by a 3-bit counter 718 fed by a 1.6 Hz clock. Resistor 722 is lower resistance than resistor 720 and resistor 724 is lower resistance than resistor 722. Thus, as the 3-bit counter 718 increments, the current through the LEDs 700, 702 will increase in a step-wise fashion generating a series of 5 second breath cues.

The 3-bit counter 718 and the 4-bit counter 714 are synchronized by a power-on-reset (POR) signal. The counters will remain synchronized since the 1/320 Hz clock is derived from the 1.6 Hz clock.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of generating light, the method comprising:
(a) generating steady light during a first period, the first period is at least twenty minutes, the first period is less than twelve hours; and
(b) generating varying light during a second period, the second period is less than one quarter of the first period, the varying light is in the form of breath cues.

2. The method of claim 1 further comprising:
(c) repeating (a) and (b).

3. The method of claim 2 wherein a breath cue has a duration of about five seconds.

4. The method of claim 2 wherein a breath cue includes a plurality of light intensities.

5. The method of claim 4 wherein the plurality of light intensities increase during the breath cue.

6. The method of claim 4 wherein two sequential breath cues are the same.

7. A light controller configured to perform the method of claim 4.

8. A lamp configured to perform the method of claim 2.

9. The lamp of claim 8 further comprising a user control, the lamp configured to adjust one of the first period, the second period, and the duration of each breath cue based on the user control.

10. The lamp of claim 8 further comprising a user control, the lamp configured to exit (b) and enter (a) when the user control is activated.

11. The lamp of claim 8 further comprising a user control, the lamp configured to exit (a) and enter (b) when the user control is activated.

12. The method of claim 1 further comprising:
c) generating an end-of-cycle indication after the second period is completed; and
d) repeating (a), (b), and (c).

13. The method of claim 1 wherein generating varying light consists of no period longer than one thirtieth of a second of generating no light.

14. The method of claim 1, wherein generating varying light includes generating varying colors of light.

15. The method of claim 14 wherein generating varying light includes generating a first color of light during a first breath cue and generating a second color of light during a second breath cue.

16. The method of claim 1 wherein a breath cue includes a plurality of sub-periods, each sub-period having a different steady light intensity, each sub-period is between a third of a second and three seconds.

17. A device configured to:
(a) generate breath cues during a first period, the first period is between one minute and thirty minutes, the breath cues are such that the total time for a complete inhale/exhale breath cycle is between six and twenty seconds;
(b) not generate breath cues during a second period, the second period is between thirty minutes and four hours; and
(c) repeat (a) and (b).

18. The device of claim 17 further comprising one or more lights that generate the breath cues.

19. A device comprising:
means for generating steady light during a first period, the first period is at least twenty minutes; and
means for generating varying light during a second period, the second period is less than one quarter of the first period, the varying light is in the form of breath cues.

20. The device of claim 19 further comprising means for adjusting the first period.
